(19) United States
(12) Patent Application Publication
(10) Pub. No.: US 2009/0087396 A1
(43) Pub. Date: Apr. 2, 2009

(54) Sweat-Absorbing Cosmetic Product and Method for the Production Thereof

(75) Inventors: Donna Hui-Ing Hwang, Leonia, NJ (US); Ralph Macchio, Sparta, NJ (US); Domnica Cernasov, Ringwood, NJ (US); Salvatore Barone, Staten Island, NY (US)

Correspondence Address: NORRIS, MCLAUGHLIN & MARCUS, P.A., 875 THIRD AVE, 18TH FLOOR, NEW YORK, NY, 10022, US

(73) Assignee: COTY B.V., Haarlem (NL)

(21) Appl. No.: 11/568,016

(22) PCT Filed: Apr. 22, 2005
(86) PCT No.: PCT/EP05/04496

§ 371 Date: Oct. 17, 2006

(30) Foreign Application Priority Data
Apr. 22, 2004 (DE) 10 2004 020 647.3

(51) Int. Cl. A61K 8/18; A61Q 15/00

(52) U.S. Cl. 424/65

(57) ABSTRACT

The invention relates to a preferably water-free sweat-absorbing cosmetic product, e.g. an antiperspirant, containing a base formulation and a sweat-absorbing complex. The invention also relates to a method for the production of said complex and said cosmetic product.

The cosmetic product of the invention includes
i) a base formulation, and
ii) a sweat-absorbing complex comprising
   (a) at least one water-absorbing component,
   (b) at least one surface-active agent, and
   (c) optionally a solvent and/or a vehicle.

The sweat-absorbing complex in the base formulation is present in the form of emulsified particles. The particles consist of a three-dimensional, water-swellable network of said at least one water-absorbing component and are at least partially covered with said at least one surface-active agent.

The invention relates to a hydrous or water-free sweat-absorbing cosmetic product, e.g. an antiperspirant, containing a base formulation and a sweat-absorbing complex. The invention also relates to a method for the production of said complex and said cosmetic product.

Cosmetic products with an anti-perspiration effect are well-known, and different classes of products can be distinguished. Deodorants eliminate unpleasant odors originating from bacterial decomposition of sweat. Deodorants therefore frequently include antibacterial substances as well as fragrant substances such as aromatic essences. Many ethereal oils have both antibacterial and fragrant properties, for which reason they are frequently used in deodorants. Products inhibiting sweat production per se, so-called antiperspirants, include adstringent agents as active substances (antiperspirant actives), especially aluminum or zirconium salts. Basically, deodorant products contain water, whereas traditional antiperspirants (including suspended adstringents in a non-polar phase such as silicone oil or mineral oil) are substantially free of water in order to prevent polymerization of the adstringent agents. This type of antiperspirant products is normally present in the form of a solid stick. Nevertheless, a new generation of transparent antiperspirants has recently been developed which has gained popularity owing to its aesthetic appearance. This new generation of antiperspirants are emulsions usually containing propylene glycol, adstringents, water and oils. However, this type of antiperspirants is less active than the traditional type due to polymerization of the adstringent agents.

Both classes of products—deodorants and antiperspirants—may additionally include substances capable of absorbing sweat, thereby imparting a fresh and dry feeling to the skin. Conventional sweat-absorbing components are, for example, polymers such as natural or chemically modified polysaccharides or polysaccharide gums, or synthetic polymers. Such polymers form three-dimensional networks capable of absorbing water.

One common problem of hydrous products (comprising emulsion type deodorants and antiperspirants) is that the water-absorbing component, once incorporated in a hydrous formulation, rapidly loses its absorptive capacity. On the other hand, water-absorbing components in traditional water-free antiperspirants show only low water or sweat absorption activity because they are surrounded by a non-polar medium, so that contact with water/sweat of the skin is prevented.

From WO 03/030853, a substantially water-free forearm product is known which includes a special water-absorbing polymer, a volatile silicone and a gelling agent, and optionally small amounts of a surfactant (to stabilize the formulation), an antiperspirant active substance or deodorizing agent, a non-volatile silicone and an emulsifier. The product is a suspension wherein the water-absorbing polymer is present in the form of dispersed particles. This formulation is not suitable for use in standard hydrous deodorant formulations because the water-absorbing polymer undergoes swelling in water, losing its water storage capacity.

The object of the present invention is to provide a sweat-absorbing cosmetic product, especially an antiperspirant product, which has increased and sustaining water absorption capacity, particularly in largely water-free, but also in water-containing (emulsion type) formulations, and overcomes the formulation problems of well-known deodorant products.

Said object is accomplished by means of a sweat-absorbing cosmetic product having the features as in claim 1. The product according to the invention includes (in parts by weight):

i) a base formulation, and
ii) a sweat-absorbing complex comprising
(a) 0.1 to 90% of at least one water-absorbing component,
(b) 0.1 to 80% of at least one surface-active agent,
(c) 0 to 50% of at least one solvent and/or at least one vehicle.

A crucial feature is that the sweat-absorbing complex in the base formulation is present in the form of emulsified particles which consist of a three-dimensional, water-swellable polymer network of said at least one water-absorbing component, and which are at least partially covered (coated) with said at least one surface-active agent.

In the complex, said at least one water-absorbing component is capable of swelling in water (but is not completely dissolved in the composition) and has a strength sufficient to form a three-dimensional gel-like network when in contact with water. In this complex, said at least one water-absorbing component is protected by a coating (cover) of said at least one surface-active agent (surfactant). The complex is intended as an additive to any base formulations, so as to provide a cosmetic product. The coating layer of said at least one surfactant uniformly suspends the absorber material within the base formulation and stabilizes the absorber particles, thereby preserving their sweat absorption capacity. The interaction between the surfactant(s) and water-absorbing component(s) is based on hydrogen bridges, the surface-active agent forming an oriented layer and acting with its lipophilic segments to stabilize the absorber material within the formulation. Following application onto the skin, the formulation containing the complex forms a thin film on the skin, during which process a reorganization of the surfactant takes place. That is, as a result of film formation, the suspending surfactants are arranged between the skin and the water-absorbing component, thus mediating contact between the skin and the water absorbing-component, which contact enables sweat absorption. This process allows rapid absorption of sweat from the skin by the water-absorbing component and also, the water absorption capacity can be maintained.

Although the complex was primarily designed for use in largely water-free antiperspirant base formulations, it is also suitable for use in water-containing formulations, e.g. in a new generation of emulsion type antiperspirants, without losing its water absorption capacity. In this event, the coating layer of said at least one surface-active agent protects the absorber material from undesirable water absorption from the aqueous phase of the formulation. In this way, only a minor proportion of the absorber material present will undergo swelling in the water of the formulation, so that most of the water storage capacity is available for sweat absorption. All in all, the high sweat absorption capacity of the absorber material is preserved for a longer period of time compared to well-known compositions.

In addition, the network of said at least one water-absorbing component provides an open three-dimensional backbone, in the cavities of which the hydrophilic segments of said at least one surfactant or other, hydrophobic materials as well can be incorporated, thereby supporting the absorption and retention of water. The incorporation of hydrophobic materials or segments prevents excessively strong hydrogen bridges of the backbone chains of the water-absorbing component, so that water/sweat can easily penetrate and push apart the absorber molecules to effect rapid hydration thereof. In other words, the inter- and intramolecular hydrogen bridges are cleaved so as to facilitate the entry of water. To this end, according to a preferred embodiment of the invention, a mixture of two or more water-absorbing components is used in the complex, at least one of them representing a hydrophilic material. Typical hydrophilic materials may serve this purpose, e.g. hydrated silicic acid (see below).

The sweat-absorbing complex and the cosmetic product of the invention have outstanding sweat absorption capacity, moisture reduction, inhibition of microorganisms and finally, substantial deodorizing benefit. The complex is suitable for incorporation in any cosmetic product, especially in water-free, but also in water-containing products such as forearm antiperspirants.

According to particularly advantageous embodiments of the invention, the sweat-absorbing complex comprises
(a) 10 to 80%, particularly 20 to 70%, and preferably about 30 to 50% of at least one water-absorbing component,
(b) 10 to 70%, particularly 20 to 60%, and preferably about 30 to 45% of at least one surface-active agent,
(c) optionally 0 to 50% of at least one solvent and/or at least one vehicle. The above ranges may vary strongly, depending on the properties of the individual components and the type of cosmetic product.

Said at least one water-absorbing component is essentially insoluble in said at least one solvent and/or vehicle but, on the other hand, is capable of swelling in water. In contact with water, it forms a three-dimensional gel-like polymer network, resulting in particles which, at least partially, are still coated with the surface-active agent. The water-absorbing component has a water absorption capacity of at least 20%, particularly at least 50%, based on its dry weight. In general, the water absorption capacity thereof is between 20 and 300% of the dry weight. The mean particle size of the water-absorbing component in the complex ranges from 0.1 to 450 μm, particularly from 10 to 200 μm. Particles with a mean particle size between 10 and 60 μm show particularly good results.

The cosmetic product containing the sweat-absorbing complex described above can be selected from a diverse group of products. This group of products comprises antiperspirants and deodorants (especially forearm products), any adstringent and/or deodorizing preparations, sticks, sprays, aerosols, creams, sunscreens, after-shaves, lotions, foundation creams, and make-ups. One advantage of the complex according to the invention is that it can be added easily to any standard preparations (base formulations) without losing its water storage capacity. In particular, such base formulations comprise water-free antiperspirant formulations which include at least one adstringent suspended in the non-polar phase, as well as hydrous emulsion type adstringent formulations of the new generation. A typical water-free antiperspirant base includes 30 to 70%, particularly 50 to 60 wt.-% of at least one non-polar solvent (e.g. silicone oil); 10 to 30%, particularly about 20 wt.-% of at least one gelling agent/thickening agent (e.g. stearyl alcohol); 10 to 30%, particularly about 20 wt.-% of at least one adstringent (e.g. aluminum and/or zirconium compounds), and 5 to 20%, particularly about 10 wt.-% of a wax (to produce a stick product).

Basically, the sweat-absorbing complex can be used in cosmetic products in a wide concentration range of from 0.05 to 99 wt.-%. Typically, the complex is included in the base formulation in a range of from 0.1 to 10 wt.-%, preferably from 1 to 6 wt.-%.

It will be appreciated that the cosmetic product can include further auxiliary substances and/or active substances (active agents), e.g. pigments, colorants, antioxidants, preservatives, other moisture-retaining substances, softeners, fragrances (aromatic essences), stabilizers, adstringents, cell turn-over promoters, cell proliferation stimulators, anti-inflammatory agents, antimicrobial agents, hormone regulators, enzyme inhibitors, UV absorbers, sunscreens and the like, and mixtures thereof.

As mentioned above, the sweat-absorbing complex according to the invention is remarkable for a coating/cover of the absorber particles by said at least one surfactant. This structure can easily be obtained by mixing said at least one water-absorbing component with said at least one surface-active agent with stirring and heat supply until a homogeneous mixture is obtained. The application of thermal energy (heat) and shear forces (by stirring) in the above step is crucial in causing rupture of inter- and intramolecular bonds within the surface-active agent and water-absorbing component and allowing generation of new physical interactions (especially hydrophobic interactions and hydrogen bridges) between said at least one surfactant and said at least one water-absorbing component in the subsequent cooling process. With no application of thermal energy and shear forces, surfactant(s) and water-absorbing component(s) merely develop very sparse physical interactions, and—following addition to a cosmetic base formulation—are unable to provide a cosmetic product with significant water absorption capacity. When using a solvent and/or a vehicle, addition thereof to the mixture of water-absorbing component and surface-active agent is effected, and stirring at elevated temperature is continued until a substantially homogeneous mixture is present.

To produce the cosmetic product, the thus prefabricated sweat-absorbing complex, already including the covered absorber particles, is simply added to a suitable base formulation, particularly to an antiperspirant base formulation.

Suitable substances for the particular ingredients of the sweat-absorbing complex will be referred to below.

### Water-Absorbing Component

Said at least one water-absorbing component is selected from any natural or synthetic polymers capable of swelling in water to form a three-dimensional gel-like network.

The water-absorbing components most preferred herein are gums or gum-like polymers. In the context of the present invention, “gum” is generally defined as any water-soluble polymer isolated from terrestrial or marine plants or microorganisms, which is capable of contributing to viscosity and/or swellability of a dispersion thereof. The water-absorbing component is preferably a gum derived from vegetable or microbial biosynthesis of terrestrial or marine organisms. Such gums essentially include repeats of monosaccharide units and have relatively high molecular weights of preferably at least 100,000 g/mol. Examples of preferred gums are guar gums (Cyamopsis tetragonolobus gum), guar derivatives, locust bean gum, scleroglucan (sclerotium gum), tamarind seed gum, dextrin gum and the like, as well as derivatives and mixtures thereof.

Another preferred class of water-absorbing components are polysaccharides of vegetable origin, essentially comprising repeats of monosaccharide units of hexoses and/or pentoses. Specific examples comprise natural celluloses, natural fibers, cellulose derivatives, microcrystalline cellulose, methylcelluloses, methoxycelluloses, hydroxyethylcelluloses, hydroxypropylcelluloses, hydroxypropylmethylcelluloses, dextrins, maltodextrins, inulin, inulin derivatives, starch, starch derivatives, derivatives and mixtures thereof.

Another group of suitable water-absorbing components is selected from chemically modified polysaccharides, particularly from the group including cellulose derivatives, starch derivatives, pectin derivatives, starch graft polymers (starch/acrylamide/sodium acrylate graft copolymers), and mixtures thereof.

According to another embodiment of the invention, said at least one water-absorbing component is selected from polyacrylate-based synthetic polymers, essentially comprising repeats of acrylic acid, acrylamide, methacrylic acid, including derivatives, copolymers or mixtures thereof. Polyacrylamides represent a specific example of such compounds.

Another group of preferred water-absorbing components is selected from silicic acids (Si(OH)₄ or SiO₂) and any types of derivatives and modifications thereof. Suitable examples comprise condensation products thereof, i.e., polysilicic acids ((SiO₂)ₘ×nH₂O), silicic anhydride (silica, SiO₂), fumed silica, hydrated silica (SiO₂×H₂O), silica gel and/or silicate esters. It was found that the water absorption rate and capacity of these silica components can be dramatically enhanced by pre-dispersing them in an oil phase prior to the production of the complex.

Obviously, it is also possible to use mixtures of the above-mentioned water-absorbing components. In a particularly preferred fashion, is envisaged to use a mixture of a component from the group of silicic acids and a component from the other groups as water-absorbing component of the complex according to the invention.

### Surface-Active Agents

As set forth above, said at least one surface-active agent assumes the function of forming a coating on the exterior surface of the particles of the water-absorbing component(s), thereby influencing the surface properties thereof. More specifically, the surface-active agents aid in retaining the polymer on the skin, they stabilize the particles in the formulation and facilitate incorporation of water. Surface-active agents comprise compounds, including monomers, dimers, trimers, oligomers and polymers, which have lipophilic as well as hydrophilic functionalities of a strength sufficient to develop affinity to both hydrophilic and lipophilic portions of the formulation. Such agents form oriented layers around the water-absorbing particles. In this way, the particles of the water-absorbing component(s) are stabilized and distributed homogeneously within the formulation and, if necessary, protected from undesirable water absorption from a hydrous base formulation.

For the purposes mentioned above, the surface-active agent has a so-called hydrophilic/lipophilic balance (HLB) of at least 4.5, particularly at least 7, preferably in a range of from 12 to 18. Very good results are obtained with surfactants having an HLB ranging from 14 to 16. The above-mentioned HLB values are predominantly selected in those cases where the complex is intended for use in water-free formulations, e.g. in traditional antiperspirants. If, on the other hand, the complex is intended for addition to hydrous formulations, e.g. emulsion type antiperspirants, lower HLB values are preferred. In the latter case, a HLB below 13, particularly in a range of from 2 to 13, preferably from 3 to 11, is selected. Surfactants having an HLB in a range of from 5 to 10 are particularly preferred in hydrous uses. The HLB is a dimensionless value developed by C. Griffin, which accounts for the relative amounts of lipophilic versus hydrophilic segments of a material. The assignment of numerical values for the HLB is based on effects of chemical groups within a molecule (D. L. Courtney, in “Surfactants in Cosmetics”, 2^{nd} edition, Marcel Dekker, Inc., New York, 1997, 128-130).

According to a preferred embodiment of the invention, a mixture of at least two surface-active agents is used in the complex. In this event, it is also possible to use surfactants with HLB values outside the above-mentioned ranges, provided an effective weighted average HLB of such a combination falls within the above-mentioned ranges. Similarly, the HLB will be selected depending on the intended use of the complex, i.e., whether in water-free or hydrous formulations.

Said at least one surface-active agent can be a non-ionic, cationic or amphoteric compound or a combination thereof. Assorted examples include fatty alcohols, ethoxylated alcohols, ethoxylated triglycerides, ethoxylated oils, monoglycerides, carboxylic esters of alkyl or alkenyl glycols, C₁-C₄₀ fatty acid esters of polyols, C₁-C₄₀ fatty acid ethers of polyols, C₁-C₄₀ fatty acid esters of alkyl or alkenyl glycols, polyglycerol esters, polyglycerol esters of C₁-C₄₀ fatty acids, hydrocarbon-derived esters, sugar esters and polyesters, alkoxylated sugar esters and polyesters, ethoxylated carboxylic esters of C₁-C₄₀ fatty acids, sorbitan or polysorbate esters of fatty acids, ethoxylated sorbitan esters of fatty acids, ethoxylated sugar ethers of fatty acids, alkoxylated derivatives of C₁-C₄₀ fatty acid esters of C₁-C₄₀ fatty alcohols, alkoxylated derivatives of C₁-C₄₀ fatty acid ethers of C₁-C₄₀ fatty alcohols, polyethylene glycol ethers, polyethylene glycol esters, ethoxylated polysiloxanes, alkyl glycosides, alkanolamides, amine oxides, fatty acid amides, alkylamidoalkylamines, alkylamines, alkylimidazolines, alkyl-substituted amino acids, and mixtures thereof. Specific examples are saccharose stearate and sorbitan sesquioleate and mixtures of thereof.

### Solvent/Vehicle

Optionally, a solvent and/or a vehicle is present in the sweat-absorbing complex. In general, this is required only in those cases where said at least one surface-active agent is a solid. In the event of a surface-active agent present in the form of a liquid, neither solvent nor vehicle are required.

Said at least one solvent and/or said at least one vehicle can be selected from the group including glycols, glycerol, polar and non-polar oils, hydrocarbons, ethers, esters, medium- and long-chain alcohols, alkoxylated alcohols, polyhydric alcohols, polyols, and mixtures thereof. Specific examples comprise propylene glycol, dipropylene glycol, ethylene glycol, glycerol, diglycerol, diacetin, triacetin, isopropyl palmitate, isododecane, isohexadecane, hydrogenated polydecene, triglycerides, mineral oil, and mixtures thereof.

Further preferred embodiments of the invention can be inferred from other features specified in the dependent claims.

The invention will now be explained in more detail in the examples with reference to the pertaining drawings wherein:

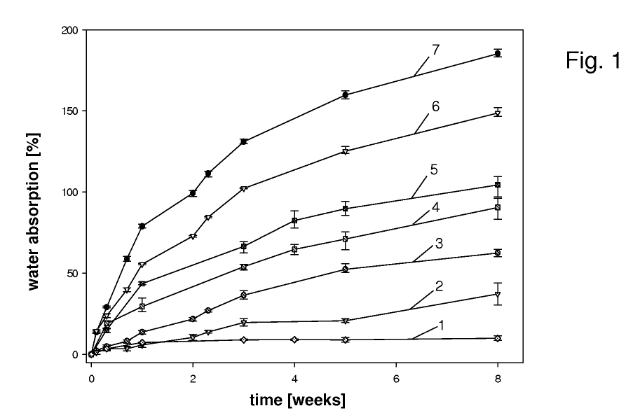

FIG. 1 shows the time profile of water absorption of various unprocessed water-absorbing components;

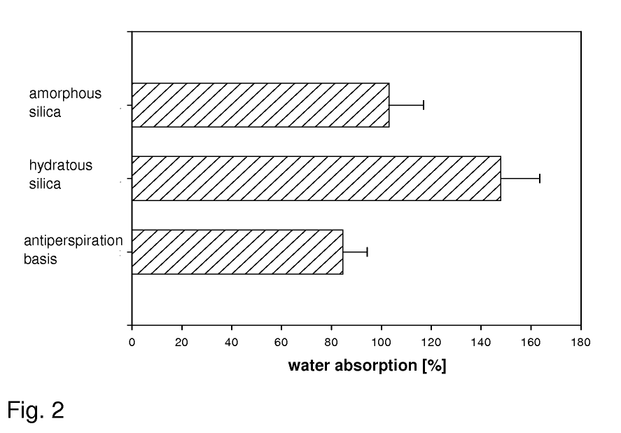

FIG. 2 illustrates the water storage capacity of unprocessed water-absorbing components in an antiperspirant base formulation;

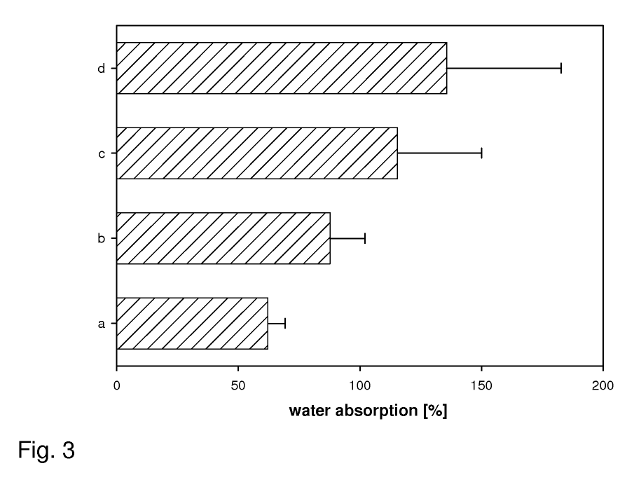

FIG. 3 illustrates the water storage capacity of sweat-absorbing complexes in an antiperspirant base formulation in accordance with a first embodiment of the invention;

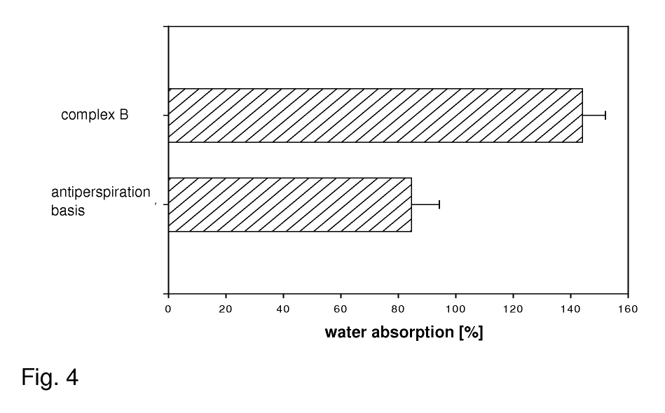

FIG. 4 illustrates the water storage capacity of sweat-absorbing complexes in an antiperspirant base formulation according to a second embodiment of the invention; and

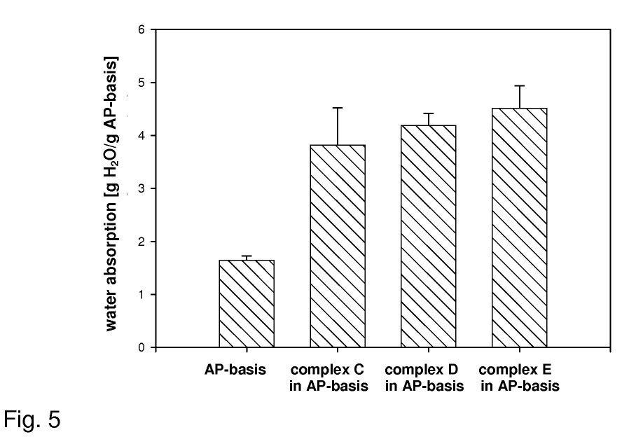

FIG. 5 illustrates the water storage capacity of sweat-absorbing complexes in an antiperspirant base formulation according to a third embodiment of the invention.

### (1) PRODUCTION OF THE SWEAT-ABSORBING COMPLEX

At least one water-absorbing component is placed in a clean, dry stainless steel tank equipped with a stirrer. A premixed combination of silicic acid or silicic acid derivative and a second water-absorbing component is preferably used. With slow stirring, the mixture is heated to a temperature between 50 and 100° C. and kept at this temperature. Subsequently, at least one surface-active agent is slowly added to the tank. While maintaining the batch temperature at 50-100° C., the mixture is stirred continuously for at least another 15 minutes, until a substantially uniform (homogeneous) mixture is obtained which does not include any undissolved raw materials. In those cases where the surface-active agent is a solid, a solvent and/or a vehicle is added to the mixture, and stirring is continued until a homogeneous mixture with no visible raw materials is present. Depending on the particular ingredients, the complex thus produced has the consistency of a paste, soft solid or hard wax.

The precise quantities of the ingredients depend on the substances being selected. The weight ratio of water-absorbing component(s) to surface-active agent(s) is 1:(0.25-2), preferably 1:(0.5-1.5). The preferred batch temperature for the process depends on the melting points of the ingredients, particularly those of the water-absorbing component and surface-active agent, and it varies between 50 and 100° C., particularly between 60 and 90° C. For most ingredients, a batch temperature between 70 and 80° C. is suitable.

### (2) PRODUCTION OF AN ANTIPERSPIRANT STICK

The sweat-absorbing complex produced according to (1) is added to a molten (water-free) antiperspirant base formulation with continuous stirring at a batch temperature of 60-90° C., particularly 70-80° C., so as to obtain a homogeneous mixture including 0.1 to 10%, particularly 1 to 6 wt.-% of said complex. The antiperspirant base formulation normally includes 30 to 70%, particularly 50 to 60 wt.-% of at least one non-polar solvent (for example, silicone fluid); 10 to 30%, particularly about 20% stearyl alcohol; 10 to 30%, particularly about 20% of at least one adstringent agent (for example, aluminum oxide salt); and 5 to 20%, particularly about 10% wax (to form a stick). The mixture is subsequently cooled to room temperature to obtain the shape of a stick.

### (3) WATER ABSORPTION BEHAVIOR OF VARIOUS WATER-ABSORBING COMPONENTS (UNPROCESSED RAW MATERIALS)

A defined quantity of each raw material (unprocessed water-absorbing component) was placed in a humidity cabinet with a relative humidity of 95-99% and incubated therein for eight weeks. The percent water absorption of each sample was calculated by differential weighing of the sample prior to and after incubation in the humidity cabinet and dividing the weight difference by the sample weight prior to incubation.

FIG. 1 shows the eight-week profile of water absorption of some examples of potential candidates for the water-absorbing component. Illustrated are the results of the following water-absorbing components: microcrystalline cellulose (curve 1), silicic anhydride (silica) (2), cellulose powder (3), polysaccharide gum (4), cellulose gum (5), starch graft polymer (6), and salt of a polyacrylic acid=sodium polyacrylate (7). The data clearly show that polysaccharide gum, cellulose gum, starch graft polymer and sodium polyacrylate (curves 4-7) each have a water storage capacity of more than 100%, based on the dry weight of the raw material. These components are therefore used with preference in the sweat-absorbing complexes according to the invention.

### (4) WATER ABSORPTION BEHAVIOR OF VARIOUS WATER ABSORBING COMPONENTS IN AN ANTIPERSPIRANT BASE FORMULATION (ANTIPERSPIRANT STICK)

The unprocessed water-absorbing component (raw material; 5 g each time) was stirred with 95 g of molten antiperspirant base formulation (50-60 wt.-% silicone fluid (cyclomethicon), 18-22% stearyl alcohol, 20% aluminum oxide salts, 10% wax, and 0.1% allantoin) and 1.5 g of an aromatic essence until to a homogeneous mixture was obtained. The mixture was cooled to room temperature to obtain the shape of a stick.

A defined quantity of each of these mixtures was placed on glass wool in a sealed container including an excess of water and subsequently incubated at 37° C. for 24 hours. A sample of pure, but aromatic essence-containing antiperspirant base formulation was incubated in the container in the same way. Following careful decanting of the excess water from the container at the end of the incubation period, the percent water absorption was determined by differential weighing of each sample prior to and after water exposure.

The results for amorphous silica and hydrated silica are illustrated in FIG. 2. In addition, the water absorption of a pure deodorant base formulation is shown as reference. The results are given as grams of absorbed water per gram of “dry” antiperspirant base with or with no silicic acid prior to incubation. The data show that the antiperspirants containing silica absorb more than 50% more water compared to a regular antiperspirant with no silica additive. It is thus demonstrated that addition of 1 wt.-% of silica provides an additional water storage capacity of about 10% compared to a regular, hydrous antiperspirant base formulation.

### (5) WATER ABSORPTION BEHAVIOR OF VARIOUS SWEAT-ABSORBING COMPLEXES IN AN ANTIPERSPIRANT BASE FORMULATION (ANTIPERSPIRANT STICK)

A sweat-absorbing complex in accordance with the present invention (complex A) was produced according to the procedure described above, using a mixture of fumed silica and scleroglucan as water-absorbing component, saccharose ester as surface-active agent, and hydrogenated polydecene as solvent. The composition of complex A is specified in Table 1.

TABLE 1

| | Complex A |
|---|---|
| Fumed silica (HDK ® N20, Wacker Chemical Corp.) | 3% |
| Scleroglucan (Alban Muller Ind.) | 30% |
| Saccharose ester (MMP, Inc.) | 40% |
| Hydrogenated polydecene (Lipo Chemicals, Inc.) | 27% |
| Sum | 100% |

Complex A, 2 g and 4 g each time, was stirred with 96.5 g and 94.5 g, respectively, of a molten antiperspirant base formulation (50-60 wt.-% silicone fluid (cyclomethicon), 18-22% stearyl alcohol, 20% aluminum oxide salts, 10% wax) and 1.5 g of aromatic essence, until a homogeneous mixture was obtained. The mixtures were cooled to room temperature to obtain the shape of a stick. Consequently, the products thus obtained included 2 and 4 wt.-%, respectively, of the overall complex and 0.66 and 1.32 wt.-%, respectively, of water-absorbing components (silica+gum).

For comparison, a mixture was produced by consecutive mixing of the individual ingredients of complex A (as specified in Table 1; with no premixing of the complex) into the molten aromatic essence-containing antiperspirant base. The individual amounts of the ingredients were selected corresponding to an overall weight percentage of complex of 2% in the product.

A defined quantity of each of these mixtures was placed on glass wool in a sealed container including an excess of water and subsequently incubated at 37° C. for 24 hours. A sample of pure, aromatic essence-containing antiperspirant base was incubated in the container in the same way as a control. After careful decanting of excess water from the container, the percent water absorption was determined by differential weighing of each sample prior to and after water exposure.

The results are illustrated in FIG. 3. Bar a represents the control (antiperspirant base). Bar b represents the comparative test wherein the ingredients were admixed individually to the base (without previous production of the complex). Bars c and d show the preparations with 2 and 4 wt.-% of premixed complex A in the antiperspirant base. The data show that the antiperspirants including 2 and 4%, respectively, of complex A have a water absorption increased by 87 and 118%, respectively, compared to the regular antiperspirant base. The same ingredients, following individual admixing, increase the water absorption by only 41% compared to the regular antiperspirant base. This example clearly demonstrates that the complex in its inventive configuration is essential in providing a base formulation with a significantly higher water storage capacity compared to the same, but individually added ingredients.

### (6) WATER ABSORPTION BEHAVIOR OF VARIOUS SWEAT-ABSORBING COMPLEXES IN AN ANTIPERSPIRANT BASE FORMULATION (ANTIPERSPIRANT STICK)

A sweat-absorbing complex in accordance with the present invention (complex B) was produced according to the procedure (1) described above, using a mixture of fumed silica and maltodextrin as water-absorbing component, saccharose ester as surface-active agent, and cyclomethicon as solvent. The composition of complex B is specified in Table 2.

TABLE 2

| | Complex B |
|---|---|
| Fumed silica (HDK ® N20, Wacker Chemical Corp.) | 12.5% |
| Maltodextrin (Grain Processing, Inc.) | 25% |
| Saccharose ester (MMP, Inc.) | 12.5% |
| Cyclomethicon (Lipo Chemicals, Inc.) | 50% |
| Sum | 100% |

4 g of premixed complex B was mixed with 94.5 g of molten antiperspirant base formulation (50-60 wt.-% silicone fluid (cyclomethicon), 18-22% stearyl alcohol, 20% aluminum oxide salts, 10% wax, 0.1% allantoin) and 1.5 g of aromatic essence until a homogeneous mixture was obtained. The mixture was cooled to room temperature to obtain the shape of a stick. Consequently, the product thus obtained included 4 wt.-% of the overall complex or 1.5 wt.-% of water-absorbing components (silica+maltodextrin).

A defined quantity of the above mixture was placed on glass wool in a sealed container including an excess of water and subsequently incubated at 37° C. for 24 hours. A sample of pure, aromatic essence-containing antiperspirant base was incubated in the container in the same way as a control. After careful decanting of excess water from the container, the percent water absorption was determined by differential weighing of each sample prior to and after water exposure.

The result is illustrated in FIG. 4. The data demonstrate that the antiperspirant base formulation with 4% of complex B increases the water absorption by more than 70% compared to the regular antiperspirant base.

### (7) WATER ABSORPTION BEHAVIOR OF VARIOUS SWEAT-ABSORBING COMPLEXES IN AN ANTIPERSPIRANT BASE FORMULATION (ANTIPERSPIRANT SPRAY)

The sweat-absorbing complexes C, D and E in accordance with the present invention were produced according to the above-described procedure (1), with the compositions in grams or wt.-% as specified in Table 3. Essentially, the complexes differ in their surface-active agents (sorbitan stearate, sorbityl laurate, saccharose ester) and, as a consequence, in their HLB values.

The sweat-absorbing complexes C, D and E were incorporated as an antiperspirant base formulation (AP base) in an antiperspirant spray concentrate so as to obtain homogeneous concentrates for antiperspirant sprays, each including 20 wt.-% sweat-absorbing complex.

A defined quantity of each sample was placed on glass wool in a sealed container. Subsequently, an excess of water was placed in the container, and the samples were incubated at 37° C. for 24 hours. As comparative example, an antiperspirant spray concentrate with no sweat-absorbing complex was treated in an analogous fashion. After careful decanting of excess water, the samples were re-weighed and the water absorption was determined by differential weighing prior to and after water exposure. The amounts of absorbed water relative to one gram of AP base are shown in FIG. 5. The data demonstrate that the antiperspirant spray products including complex C, D or E have a water absorption increased by 133%, 155 and 175%, respectively, compared to the pure AP base.

TABLE 3

| | Complex C | Complex D | Complex E |
|---|---|---|---|
| Silicic anhydride (silica; | 2.5 | 2.5 | 2.5 |
| Wacker Chemical Corp.) | | | |
| Tara gum (TIC Gums) | 37.5 | 37.5 | 37.5 |
| Natural cotton | 0.2 | 0.2 | 0.2 |
| Saccharose ester (MMP. Inc.), | 5 | 15 | — |
| HLB~15 | | | |
| Sorbitan stearate and | 30 | 15 | 30 |
| sorbityl laurate (HLB ~6) | | | |
| Hydrogenated polydecene | 24.8 | 29.8 | 29.8 |
| (Lipo Chemicals, Inc.) | | | |
| Sum | 100 | 100 | 100 |

1. A sweat-absorbing cosmetic product, said product containing comprising
i) a base formulation, and
ii) a sweat-absorbing complex comprising
(a) 0.1 to 90% of at least one water-absorbing component,
(b) 0.1 to 80% of at least one surface-active agent,
(c) 0 to 50% of at least one solvent and/or at least one vehicle,
wherein the sweat-absorbing complex forms particles of a three-dimensional water-swellable network of said at least one water-absorbing component, which particles are at least partly coated by said at least one surface-active agent and emulsified in the base formulation.

2. The cosmetic product according to claim 1, wherein the sweat-absorbing complex comprises
(a) 10 to 80 wt.-% of said at least one water-absorbing component,
(b) 10 to 70 wt.-% of said at least one surface-active agent, and
(c) 0 to 50 wt.-% of said at least one solvent and/or vehicle.

3. The cosmetic product according to claim 2, wherein the sweat-absorbing complex comprises
(a) 20 to 70 wt.-%, particularly about 30 to 50 wt.-%, of said at least one water-absorbing component,
(b) 20 to 60 wt.-%, particularly about 30 to 45 wt.-%, of said at least one surface-active agent, and
(c) 0 to 50 wt.-% of said at least one solvent and/or said at least one vehicle.

4. The cosmetic product according to claim 1, characterized in that said at least one water-absorbing component has a water absorption capacity of at least 20%, particularly at least 50%, based on the dry weight thereof.

5. The cosmetic product according to claim 1, characterized in that said at least one water-absorbing component forms particles of a three-dimensional polymeric network when in contact with water.

6. The cosmetic product according to claim 5, characterized in that said at least one water-absorbing component forms particles with a mean particle size ranging from 0.1 to 450 μm, particularly from 10 to 200 μm, and preferably from 10 to 60 μm.

7. The cosmetic product according to claim 1, characterized in that said at least one water-absorbing component is essentially insoluble in said at least one solvent and/or vehicle.

8. The cosmetic product according to claim 1, characterized in that said at least one water-absorbing component is selected from gums from vegetable or microbial biosynthesis, which essentially comprise repeats of monosaccharide units and have a molecular weight of at least 100,000 g/mol, particularly selected from the group including guar gum, guar derivatives, locust bean gum, scleroglucan, tamarind seed gum, dextrin gum, derivatives and mixtures thereof.

9. The cosmetic product according to claim 1, characterized in that said at least one water-absorbing component is selected from polysaccharides of vegetable origin, essentially comprising repeats of monosaccharide units of hexoses and/or pentoses, particularly selected from the group including natural celluloses, natural fibers, cellulose derivatives, microcrystalline cellulose, methylcelluloses, methoxycelluloses, hydroxyethylcelluloses, hydroxypropylcelluloses, hydroxypropylmethylcelluloses, dextrins, maltodextrins, inulin, inulin derivatives, starch, starch derivatives, derivatives and mixtures thereof.

10. The cosmetic product according to claim 1, characterized in that said at least one water-absorbing component is selected from chemically modified polysaccharides, particularly from the group including cellulose derivatives, starch derivatives, pectin derivatives, starch graft copolymers, and mixtures thereof.

11. The cosmetic product according to claim 1, characterized in that said at least one water-absorbing component is selected from polyacrylate-based synthetic polymers, particularly comprising repeats of acrylic acid, acrylamide, methacrylic acid, derivatives or mixtures thereof, particularly polyacrylamide.

12. The cosmetic product according to claim 1, characterized in that said at least one water-absorbing component is selected from silicic acids and derivatives and modifications thereof, particularly from the group including polysilicic acids ((SiO₂)ₘ×nH₂O), silica (SiO₂), fumed silica, hydrated silica (SiO₂×H₂O), silica gel and/or silicate esters.

13. The cosmetic product according to claim 1, characterized in that, if the product is essentially water-free, said at least one surface-active agent has a hydrophilic/lipophilic balance (HLB) of at least 4.5, particularly at least 7, preferably in a range of from 12 to 18, more preferably from 14 to 16.

14. The cosmetic product according to claim 13, characterized in that, if the product is essentially water-free, the complex comprises a mixture of at least two surface-active agents having an effective weighted average hydrophilic-lipophilic balance (HLB) of at least 4.5, particularly of at least 7, preferably in the range of from 12 to 18, more preferably from 14 to 16.

15. The cosmetic product according to claim 1, characterized in that, if the product is a hydrous product, said at least one surface-active agent or a mixture of at least two surface-active agents has a hydrophilic-lipophilic balance (HLB) in the range between 2 and 13, preferably from 3 to 11, and preferably from 5 to 10.

16. The cosmetic product according to claim 1, characterized in that said at least one surface-active agent is a non-ionic, cationic or amphoteric compound or a mixture thereof, particularly selected from the group including fatty alcohols, ethoxylated alcohols, ethoxylated triglycerides, ethoxylated oils, monoglycerides, carboxylic esters of alkyl or alkenyl glycols, C₁-C₄₀ fatty acid esters of polyols, C₁-C₄₀ fatty acid ethers of polyols, C₁-C₄₀ fatty acid esters of alkyl or alkenyl glycols, polyglycerol esters, polyglycerol esters of C₁-C₄₀ fatty acids, hydrocarbon-derived esters, sugar esters and polyesters, alkoxylated sugar esters and polyesters, ethoxylated carboxylic esters of C₁-C₄₀ fatty acids, sorbitan or polysorbate esters of fatty acids, ethoxylated sorbitan esters of fatty acids, ethoxylated sugar ethers of fatty acids, alkoxylated derivatives of C₁-C₄₀ fatty acid esters of C₁-C₄₀ fatty alcohols, alkoxylated derivatives of C₁-C₄₀ fatty acid ethers of C₁-C₄₀ fatty alcohols, polyethylene glycol ethers, polyethylene glycol esters, ethoxylated polysiloxanes, alkyl glycosides, alkanolamides, amine oxides, fatty acid amides, alkylamidoalkylamines, alkylamines, alkylimidazolines, alkyl-substituted amino acids, and mixtures thereof.

17. The cosmetic product according to claim 1, characterized in that said at least one solvent and/or said at least one vehicle is selected from the group including glycols, glycerol, polar and non-polar oils, hydrocarbons, ethers, esters, medium- and long-chain alcohols, alkoxylated alcohols, polyhydric alcohols, polyols, and mixtures thereof, particularly from the group including propylene glycol, dipropylene glycol, ethylene glycol, glycerol, diglycerol, diacetin, triacetin, isopropyl palmitate, isododecane, isohexadecane, hydrogenated polydecene, triglycerides, mineral oil, and mixtures thereof.

18. The cosmetic product according to claim 1, characterized in that the product is selected from the group including antiperspirants, deodorants, any adstringent and/or deodorizing preparations, sticks, sprays, aerosols, creams, sunscreens, after-shaves, lotions, foundation creams, and make-ups.

19. The cosmetic product according to claim 1, characterized in that the sweat-absorbing complex has a weight percentage in the product of from 0.05 to 99 wt.-%, particularly from 0.1 to 10 wt.-%, and preferably from 1 to 6 wt.-%.

20. The cosmetic product according to claim 1, further comprising auxiliary substances and/or active agents selected from the group including pigments, colorants, antioxidants, preservatives, other moisture-retaining substances, softeners, fragrances, stabilizers, adstringents, cell turn-over promoters, cell proliferation stimulators, anti-inflammatory agents, antimicrobial agents, hormone regulators, enzyme inhibitors, UV absorbers, sunscreens, and mixtures thereof.

21. The cosmetic product according to claim 1, characterized in that the product is an essentially water-free antiperspirant containing 0.1 to 10 wt.-% of said sweat-absorbing complex in an antiperspirant base formulation.

22. The cosmetic product according to claim 21, wherein the antiperspirant base formulation comprises 30-70 wt.-%, particularly 50-60 wt.-% of at least one non-polar solvent, 5-20 wt.-%, particularly about 10 wt.-% of a wax, 10-30%, particularly about 20 wt.-% of at least one gelling or thickening agent, and 10-30 wt.-%, particularly about 20 wt.-% of at least one adstringent.

23. The cosmetic product according to claim 22, wherein the solvent is silicone oil, the gelling agent is stearyl alcohol, and the adstringent is an aluminum and/or zirconium salt, particularly an aluminum oxide salt and/or a zirconium oxide salt.

24. A method for the production of a sweat-absorbing complex for a cosmetic product according to any of claim 1, wherein said at least one water-absorbing component and said at least one surface-active agent are mixed with stirring and heat supply until a substantially homogeneous mixture is obtained.

25. The method according to claim 24, wherein said at least one solvent and/or said at least one vehicle is added to the mixture of said at least one water-absorbing component and said at least one surface-active agent with stirring and heat with supply until a substantially homogeneous mixture is obtained.

26. The method according to claim 24 or 25, wherein the steps of mixing said at least one water-absorbing component and said at least one surface-active agent and/or of adding said at least one solvent and/or said at least one vehicle are conducted at a temperature between 50 and 100° C., particularly between 60 and 90° C., preferably between 70 and 80° C.

27. A method for the production of a cosmetic product comprising
i) a base formulation, and
ii) a sweat-absorbing complex comprising
(a) 0.1 to 90% of at least one water-absorbing component,
(b) 0.1 to 80% of at least one surface-active agent,
(c) 0 to 50% of at least one solvent and/or at least one vehicle,
wherein the sweat-absorbing complex forms particles of a three-dimensional water-swellable network of said at least one water-absorbing component, which particles are at least partly coated by said at least one surface-active agent and emulsified in the base formulation, the method comprising the steps of producing a sweat-absorbing complex wherein said at least one water-absorbing component and said at least one surface-active agent are mixed with stirring and heat supply until a substantially homogeneous mixture is obtained and mixing the sweat-absorbing complex with a base formulation.

28. The method according to claim 27, wherein the base formulation is an antiperspirant formulation containing at least one adstringent, particularly a substantially water-free antiperspirant formulation.
